(19) United States
(12) Patent Application Publication
(10) Pub. No.: US 2025/0169743 A1
(43) Pub. Date: May 29, 2025

(54) AUDITORY AND OLFACTORY MAPPING SYSTEM

(71) Applicant: Zayed University, Dubai (AE)

(72) Inventor: Kaustuv Kanti Ganguli, Abu Dhabi (AE)

(73) Assignee: Zayed University, Dubai (AE)

(21) Appl. No.: 18/957,699

(22) Filed: Nov. 23, 2024

(30) Foreign Application Priority Data
Nov. 24, 2023 (IN) 202331079807

(51) Int. Cl. A61B 5/00

(52) U.S. Cl. CPC A61B 5/4011; A61B 5/7264; A61B 5/7405

(57) ABSTRACT

A method includes receiving, by a computing system, olfactory information. The method includes converting, by the computing system, the olfactory information to auditory information.

### BACKGROUND

Synesthesia is a stimulation of one sensory or cognitive pathway which leads to automatic, involuntary experiences in a second sensory or cognitive pathway. Human cognition through neuro-transmission from different sensory organs bears a similar form of transduction signals to the different areas of the brain. Synesthesia is used as a technique for providing music therapy, aroma therapy and similar therapies. Synesthesia also helps a person to have better perception, better memories, better creativity and many more things.

Synesthetic refers to multi-sensory experiments in the genres of visual music, music visualization, audiovisual art, abstract film, and intermedia. Distinct from neuroscience, the concept of synesthesia is regarded as the simultaneous perception of multiple stimuli in one gestalt experience. As such, it is the mixing of senses: when one sense is activated, another unrelated sense is activated at the same time.

While synesthesia provides valuable insights, there is a lack of practical applications that systematically integrate auditory and olfactory stimuli for therapeutic purposes. Also, existing methods lack integration with other sensory modalities, limiting the potential for a comprehensive therapeutic experience that engages multiple senses simultaneously.

### BRIEF DESCRIPTION OF DRAWINGS

FIG. 1 is a diagram of example block diagram;

FIG. 2 is a diagram of an example flowchart;

FIG. 3 is a diagram of an example flowchart;

FIG. 4 is a diagram of an example flowchart;

FIG. 5 is a networking environment; and

FIG. 6 is a diagram of a computer.

### DETAILED DESCRIPTION OF PREFERRED EMBODIMENTS

The following detailed description refers to the accompanying drawings. The same reference numbers in different drawings may identify the same or similar elements.

Systems, devices, and/or methods described herein are for one or more processes that convert auditory information (e.g., music, sounds, etc.) into olfactory information (e.g., smell); and, for one or more processes that convert olfactory information into auditory information. In embodiments, artificial intelligence (AI) and spectrum analysis may be used to make such conversions (e.g., mapping) between sound and smell, and vice versa.

By leveraging AI-based mapping, the systems, devices, and/or methods described herein create a seamless and dynamic synesthetic experience that can be tailored to individual profiles and preferences. According such mapping between auditory and olfactory information can (a) enhance therapy by amplifying the therapeutic effects, providing a more comprehensive and engaging treatment modality via combining music and aroma, (b) provide customization which allows for therapeutic stimuli that are precisely aligned with individual needs and preferences, optimizing the overall effectiveness; and/or (c) provide new avenues in healthcare, including stress reduction, pain management, cognitive enhancement, and rehabilitation.

In embodiments, a note of music (music note) refers to a symbolic representation of a sound, indicating its pitch and duration. Each music note on the musical scale corresponds to a specific frequency and time value, forming the building blocks of melodies and harmonies. In embodiments, a note of fragrance (fragrance note or olfactory note) is an individual scent or aromatic element within a perfume. In embodiments, perfumes can be composed of top, middle, and base notes, each contributing distinct olfactory qualities. In embodiments, the combination of these fragrance notes creates the perfume's overall fragrance profile, unfolding over time as the scent evolves.

In embodiments, auditory-olfactory synesthesia is where stimulation of one sensory pathway (e.g., hearing music) leads to involuntary experiences in another sensory domain (e.g., perceiving specific smells). In embodiments, aroma spectrum is an apparatus that provides a range of particular aroma within a defined range as prescribed in the standard aroma data sheets. In embodiments, coherence is a verification that the auditory representation maintains the overall character and structure of the original fragrance profile.

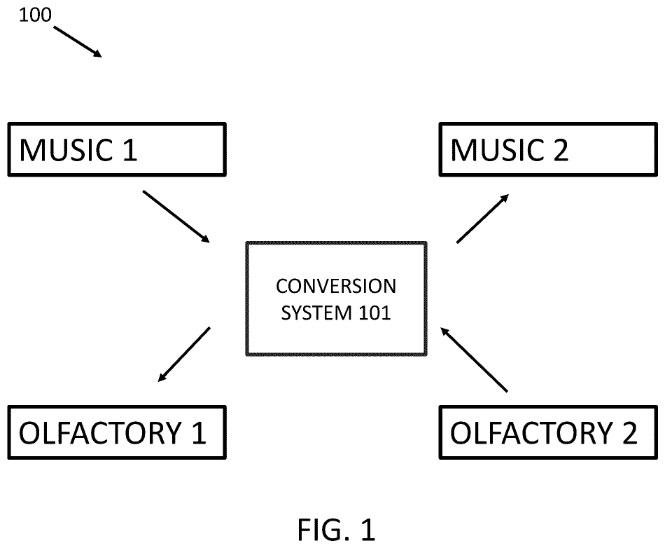

FIG. 1 is an example block diagram 100 describing an overview of the process of converting auditory information into olfactory information, and vice versa. As shown in FIG. 1, in this non-limiting example, Music 1 is sent to mapping system 101. Music 1 may include electronic information associated with classical music. When mapping system 101 receives Music 1, mapping system 101 converts the music notes (electronic information of the music notes) into olfactory notes (electronic information of olfactory notes). Mapping system 101 then generates a smell (Olfactory 1) based on the olfactory notes that can be sensed by a user of the device that sent Music 1 to mapping system 101.

Also shown in FIG. 1, Olfactory 2 is sent to mapping system 101. In this non-limiting example, Olfactory 2 is sent from another user device to mapping system 101 and Olfactory 2 is associated with a strong musk smell. In this non-limiting example, Olfactory 2 may be converted into fragrance notes (electronic information that is sent to mapping system 101. In this non-limiting example, mapping system 101 receives the fragrance notes associated with Olfactory 2 and electronically converts these fragrance notes into other electronic information which is music notes (Music 2). In this non-limiting example, Music 2 is jazz music and is sent to another user device.

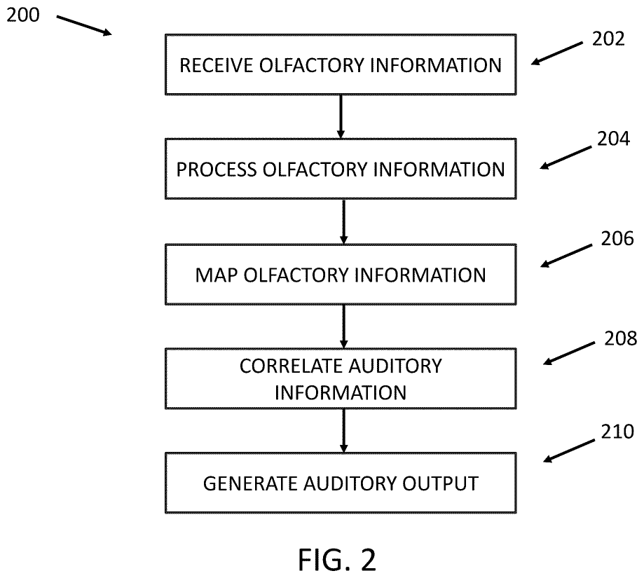

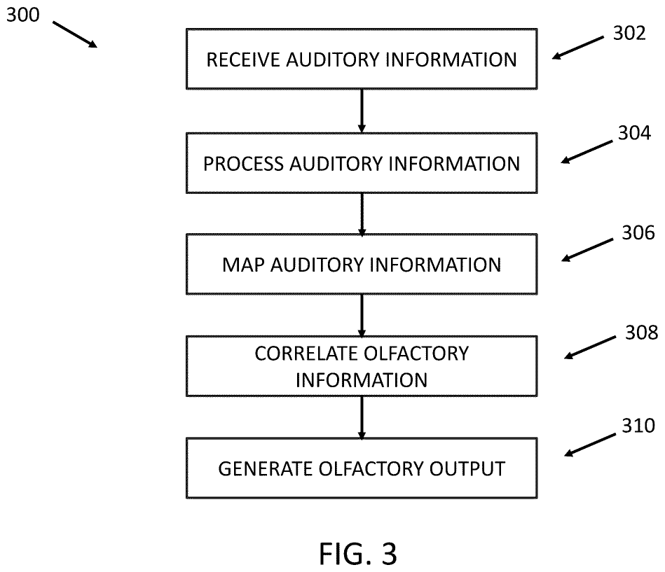

FIG. 2 is an example flowchart 200. In embodiments, flowchart 200 may be performed by a mapping system (e.g., mapping system 101) that can convert olfactory electronic information (fragrance notes) into auditory electronic information (music notes). In embodiments, the conversion of auditory electronic information (music notes) into olfactory electronic information (fragrance notes) is described in FIG. 3.

As shown in FIG. 2, at step 202, the mapping system receives olfactory information. In embodiments, olfactory information may consist of one or more fragrance notes. In embodiments, the mapping system may have one or more sensors that can be used to determine components of the received smell associated with the olfactory information. At step 204, the mapping system processes the olfactory information. In embodiments, processing the olfactory information includes deconstructing (e.g., dismantle, segregate) the olfactory information into different fragrance notes. In embodiments, the mapping system deconstructs the olfactory information into each type of fragrance note and also the frequency of each type of fragrance note. In embodiments, the mapping system includes one or more signal processing algorithms to extract electronic information about different aromatic profiles.

For example, a smell may include three aromatic profiles. In this non-limiting example, a smell may include three aromatic profiles such as floral, citrus, and woody. In this non-limiting example, one aromatic profile may have a frequency of 440 Hz (corresponding to the musical note A4) and another aromatic profile may have another frequency of 261.6 Hz (corresponding to the musical note C4).

At step 206, the mapping system maps the olfactory information, based on the deconstructed fragrance notes, to a sound. In embodiments, the mapping system may include an AI model that determines what sounds are associated with the deconstructed fragrance notes and/or the combination of the fragrance notes (the olfactory electronic information). In embodiments, the AI model may map the sound to the olfactory information in real-time. In embodiments, the mapping system (using the AI model) may determine pitch, intensity, and emotional factors when determining the sound. In embodiments, the mapping system maps the olfactory information, based on the deconstructed fragrance notes, to a sound. In embodiments, the mapping system may include an AI model that determines what sounds are associated with the deconstructed fragrance notes and/or the combination of the fragrance notes (the olfactory electronic information).

In embodiments, the AI model makes this determination by (a) analyzing the chemical structure of each fragrance note using graph neural networks (GNNs) to understand the relationships between atoms and bonds; (b) comparing the analyzed structures to a pre-trained database of fragrance-sound mappings, which has been developed using machine learning techniques on a large dataset of known fragrance-sound associations; (c) utilizing a principal odour map that clusters similar scents together based on their molecular structures and perceived characteristics; (d) applying algorithms that translate the olfactory characteristics (such as intensity, volatility, and chemical composition) into corresponding sound properties (such as pitch, timbre, and duration); and/or (e) considering emotional factors associated with both the fragrance notes and potential sound outputs which is based on electronic data from human sensory panels and psychological studies. In embodiments, the AI model may map the sound to the olfactory information in real-time. In embodiments, the mapping system (using the AI model) may determine pitch, intensity, and emotional factors when determining the sound.

At step 208, the mapping system may correlate the auditory information. In embodiments, the mapping system may adjust the auditory information based on past user feedback, environmental factors, the source of the smell, the location of the user, and/or other factors. In embodiments, this may include real-time adjustments to different sound factors intensity and using different weighing factors to adjust/correlated the generated auditory information.

At step 210, the mapping system may generate auditory output. In embodiments, the mapping system may an output device (such as a speaker) that generates the auditory output. In alternate embodiments, the mapping system may send the auditory output to another device (such as a smartphone, laptop, etc.) that then outputs the generated sound that is based on the olfactory information provided in step 202.

FIG. 3 is an example flowchart 300. In embodiments, flowchart 300 may be performed by a mapping system (e.g., mapping system 101) that can convert auditory electronic information (music notes) into olfactory electronic information (fragrance notes that can be combined together as a smell).

At step 302, the mapping system may receive auditory electronic information. In embodiments, the auditory electronic information may include one or more music notes. At step 304, the mapping system processes the auditory electronic information. In embodiments, processing the auditory electronic information includes deconstructing (e.g., dismantle, segregate) the auditory electronic information into different music notes. In embodiments, the mapping system deconstructs the auditory electronic information into different types of music note and also the frequency of each type of music note. In embodiments, the mapping system includes one or more signal processing algorithms to extract electronic information about music notes such as pitch, intensity, and any emotional characteristics.

At step 306, the mapping system maps the auditory electronic information to olfactory electronic information. In embodiments, the mapping system may include an AI model that determines what smells are associated with the deconstructed music notes and/or the combination of the music notes (the auditory electronic information). In embodiments, the AI model may map the smell the auditory electronic information in real-time. In embodiments, the mapping system (using the AI model) may determine pitch, intensity, and emotional factors when determining the sound.

At step 308, the mapping system correlates the olfactory information. In embodiments, the mapping system may adjust the olfactory electronic information based on past user feedback, environmental factors, the source of the sound, the location of the user, and/or other factors. In embodiments, this may include real-time adjustments to different olfactory factors intensity and using different weighing factors to adjust/correlated the generated olfactory information.

At step 310, the mapping system generates the olfactory information. In embodiments, the mapping system may send the olfactory electronic information to a device that mixes different smells and can convert the electronic information into a smell that can be sensed by a user of the mapping system.

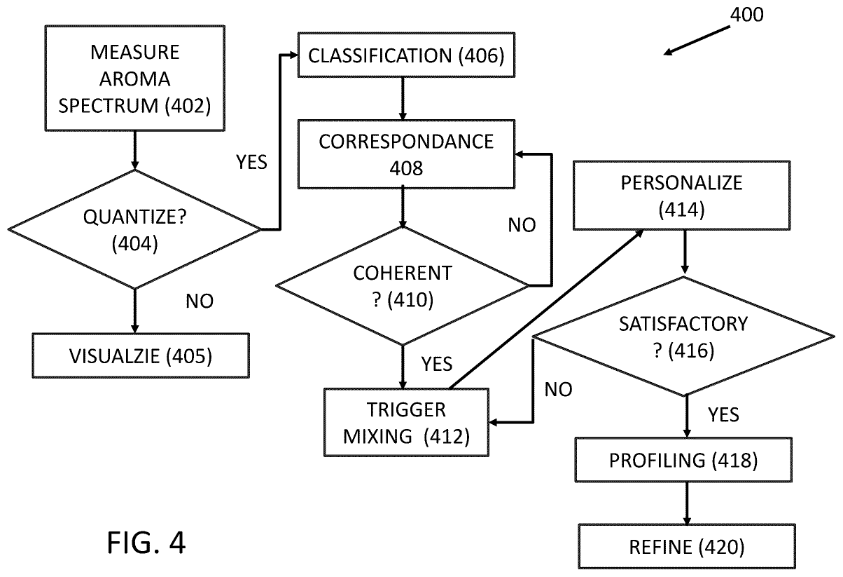

FIG. 4 describes example flowchart 400. In embodiments, flowchart 400 may be performed by a mapping system. In embodiments, example flowchart 400 is for receiving olfactory information and converting the olfactory information into auditory electronic information. In embodiments, example flowchart 400 can also be used to receive auditory information and convert the auditory information into olfactory electronic information. Thus, in that instance, each step of flowchart 400 would have instances of olfactory information replaced with auditory information, such that the input is auditory information and the output is olfactory information. In embodiments, flowchart 400 integrates sensory data processing, machine learning classification, and user-centric refinement to create a comprehensive auditory-olfactory mapping solution, potentially revolutionizing fields such as perfumery, food science, and multimedia experiences.

At step 402, mapping system conducts a spectrum measurement. In embodiments, the aroma spectrum is an apparatus that provides a range of 15 particular aroma within a defined range as prescribed in the standard aroma data sheets. At step 402 the mapping system receives the olfactory information based on the aroma from the spectrum into specific category. For example, if the rose aroma is determined with a standard intensity range of 15-20 on a predefined olfactory intensity scale (where 0 represents no detectable scent and 30 represents the highest intensity), then this is further characterized into a specific category with respective parameters. The intensity range helps classify the strength of the aroma, which assist in accurate mapping to auditory information.

At step 404, during quantization of octave folded bins, the mapping system discretizes continuous aroma spectrum data into manageable, musical-theory-inspired 12-bin structure, enabling efficient processing and mapping At step 404, the mapping system discretizes continuous aroma spectrum data into a manageable, musical-theory-inspired 12-bin structure, enabling efficient processing and mapping. This discretization process works as follows: (i) equal-width binning: the system divides the full range of the aroma spectrum data into 12 equal-width intervals, mirroring the 12 semitones in an octave; (ii) octave folding: the system applies the concept of octave equivalence from music theory. Just as musical notes repeat in higher octaves, the aroma data is “folded” so that similar scents in different intensity ranges are grouped together; (iii) boundary adjustment: the system fine-tunes bin boundaries to ensure that closely related aroma components fall within the same bin, improving the coherence of the discretization; (iv) data assignment: each aroma data point is assigned to one of the 12 bins based on its spectral characteristics; and/or (v) normalization: the system normalizes the data within each bin to ensure consistent representation across the spectrum.

If the aroma spectrum data is successfully discretized into 12 distinct bins, and alignment with musical octave principles (404—YES), then the mapping system proceeds to classification at step 406. However, if the aroma spectrum data cannot be properly quantized (404—NO), possibly due to insufficient resolution or irregular spectrum distribution, the system proceeds to step 405 for further analysis.

At step 405, the mapping system conducts further analysis, which may include (a) adaptive binning: adjusting the number or width of bins based on the data distribution; (b) feature extraction: applying signal processing techniques to extract more distinguishable features from the spectrum; (c) dimensionality reduction: using techniques like principal component analysis to simplify the data structure while preserving essential information; and/or (d) alternative discretization methods: employing other techniques such as equal-frequency binning or clustering-based approaches if equal-width binning is unsuitable. Accordingly, this refined discretization process ensures that the aroma spectrum data is effectively quantized into a structure that facilitates efficient mapping to auditory elements, even when dealing with complex or irregular spectral distributions.

At step 406, mapping system conducts classification. In embodiments, classification includes the mapping system using advanced logical intelligence and machine learning techniques to categorize complex aroma profiles into distinct, recognizable groups, enhancing accuracy of subsequent mapping. At step 408, the mapping system conducts correspondence. In embodiments, correspondence includes the mapping system establishing a relationship between specific olfactory notes and auditory elements by matching aroma characteristics (e.g., intensity, volatility, chemical composition) and sound properties (e.g., pitch, timbre, duration). In embodiments, the correspondence process utilizes machine learning models trained on extensive datasets of aroma-sound pairings.

At step 410, the mapping system determines whether the mapping is coherent. In embodiments, determining coherence, the mapping system determines consistency between mapped olfactory and auditory elements, maintaining the integrity of the sensory experience. In embodiments, the coherence check ensures that the mapped relationships between aromas and sounds make logical and perceptual sense. This includes verifying that the auditory representation maintains the overall character and structure of the original scent profile. In embodiments, this may include determining how similar aromas are mapped to related sounds across different samples or users. If inconsistencies or perceptual mismatches are detected in the mapping (410—NO), then the mapping system reviews the mapping process and returns back to step 408. If the mapped relationship between olfactory and auditory elements is logically consistent and perceptually meaningful (410—YES), then the process flow moves to step 412.

At step 412, the mapping system includes a trigger mixing device with mapped notes. At 414, the mapping system conducts personalization. In embodiments, personalization includes the mapping system incorporating user-specific sensory thresholds and preferences to tailor the aroma-sound mappings, resulting in individualized and more meaningful cross-modal experiences. In embodiments, at step 414, the mapping system tailors the mapping process to individual user preferences or physiological responses. In embodiments, the mapping system may adjust the sensitivity or weighting of certain aroma-sound correlations based on personal sensory thresholds, cultural backgrounds, or aesthetic preferences. Accordingly, this ensures the final output is optimized for each user's unique perceptual experience. Thus, at step 412, the mapping system may generate a sound that can be heard by a user of the system. At step 414, a determination is made by the user if the sound is satisfactory.

If the user sends a input (e.g., keyboard input, voice input, touchscreen input, etc.) that they are not satisfied with the current mapping (416—NO), then the mapping system returns to step 412 for further adjustments. If the personalized mapping meets the expectations and sensory preferences (416—YES), then the mapping system conducts step 418 to evaluation with standardization with iterative refinements, improving the overall quality and user experience of the system. At step 420, the mapping system includes a review of mapping which includes a feedback loop that allows for classification and correspondence process refinement.

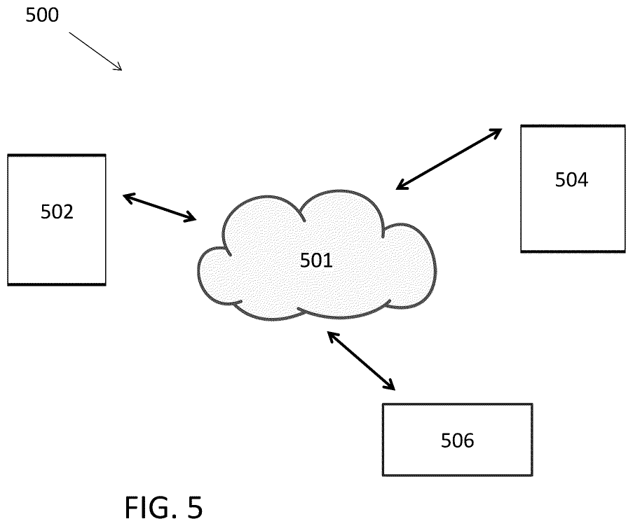

FIG. 5 is a diagram of example environment 500 in which systems, devices, and/or methods described herein may be implemented. FIG. 5 shows network 501, user device 502, user device 504, and mapping system 506.

Network 501 may include a local area network (LAN), wide area network (WAN), a metropolitan network (MAN), a telephone network (e.g., the Public Switched Telephone Network (PSTN)), a Wireless Local Area Networking (WLAN), a WiFi, a hotspot, a Light fidelity (LiFi), a Worldwide Interoperability for Microware Access (WiMax), an ad hoc network, an intranet, the Internet, a satellite network, a GPS network, a fiber optic-based network, and/or combination of these or other types of networks. Additionally, or alternatively, network 500 may include a cellular network, a public land mobile network (PLMN), a second generation (2G) network, a third generation (3G) network, a fourth generation (4G) network, a fifth generation (5G) network, and/or another network.

In embodiments, network 501 may allow for devices describe any of the described figures to electronically communicate (e.g., using emails, electronic signals, URL links, web links, electronic bits, fiber optic signals, wireless signals, wired signals, etc.) with each other so as to send and receive various types of electronic communications.

User device 502 and/or 504 may include any computation or communications device that is capable of communicating with a network (e.g., network 501). For example, user device 502 and/or user device 504 may include a radiotelephone, a personal communications system (PCS) terminal (e.g., that may combine a cellular radiotelephone with data processing and data communications capabilities), a personal digital assistant (PDA) (e.g., that can include a radiotelephone, a pager, Internet/intranet access, etc.), a smart phone, a desktop computer, a laptop computer, a tablet computer, a camera, a personal gaming system, a television, a set top box, a digital video recorder (DVR), a digital audio recorder (DUR), a digital watch, a digital glass, or another type of computation or communications device.

User device 502 and/or 504 may receive and/or display content. The content may include objects, data, images, audio, video, text, files, and/or links to files accessible via one or more networks. Content may include a media stream, which may refer to a stream of content that includes video content (e.g., a video stream), audio content (e.g., an audio stream), and/or textual content (e.g., a textual stream). In embodiments, an electronic application may use an electronic graphical user interface to display content and/or information via user device 502 and/or 504. User device 502 and/or 504 may have a touch screen and/or a keyboard that allows a user to electronically interact with an electronic application. In embodiments, a user may swipe, press, or touch user device 502 and/or 504 in such a manner that one or more electronic actions will be initiated by user device 502 and/or 504 via an electronic application. User device 502 and/or 504 may receive electronic information from antenna 506 and generate and display graphs such as those described in the figures above.

User device 502 and/or 504 may include a variety of applications, such as, for example, an e-mail application, a telephone application, a camera application, a video application, a multimedia application, a music player application, a visual voice mail application, a contacts application, a data organizer application, a calendar application, an instant messaging application, a texting application, a web browsing application, a blogging application, and/or other types of applications (e.g., a word processing application, a spreadsheet application, etc.). In embodiments, user device 502 and/or 504 may be used to generate graphs (such as those described in FIGS. 3 and 4) to model various features of the device described in FIG. 2.

Mapping system 506 may include any computation or communications device that is capable of communicating with a network (e.g., network 501). In embodiments, mapping system 506 may receive auditory information (e.g., music, spoken words, sounds, etc.) and convert the auditory information into olfactory information (e.g., smell). In embodiments, mapping system 506 may be similar to mapping system 101 and have the features of mapping system 101 as described above. In embodiments, mapping system 506 may be similar to the mapping system described in FIGS. 2, 3, and 4. In embodiments, user device 502 and/or 504 may have an electronic application that sends electronic information (such as auditory and/or olfactory information) to mapping system 506.

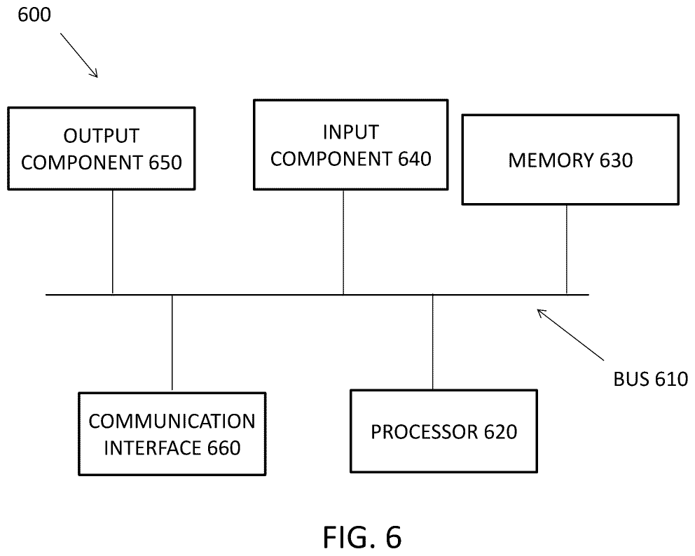

FIG. 6 is a diagram of example components of a device 600. Device 600 may correspond to user device 502, or user device 504. Alternatively, or additionally, user device 502 and user device 504 may include one or more devices 600 and/or one or more components of device 600.

As shown in FIG. 6, device 600 may include a bus 610, a processor 620, a memory 630, an input component 640, an output component 650, and a communications interface 660. In other implementations, device 600 may contain fewer components, additional components, different components, or differently arranged components than depicted in FIG. 6. Additionally, or alternatively, one or more components of device 600 may perform one or more tasks described as being performed by one or more other components of device 600.

Bus 610 may include a path that permits communications among the components of device 600. Processor 620 may include one or more processors, microprocessors, or processing logic (e.g., a field programmable gate array (FPGA) or an application specific integrated circuit (ASIC)) that interprets and executes instructions. Memory 630 may include any type of dynamic storage device that stores information and instructions, for execution by processor 620, and/or any type of non-volatile storage device that stores information for use by processor 620. Input component 640 may include a mechanism that permits a user to input information to device 600, such as a keyboard, a keypad, a button, a switch, voice command, etc. Output component 650 may include a mechanism that outputs information to the user, such as a display, a speaker, one or more light emitting diodes (LEDs), etc.

Communications interface 660 may include any transceiver-like mechanism that enables device 600 to communicate with other devices and/or systems. For example, communications interface 660 may include an Ethernet interface, an optical interface, a coaxial interface, a wireless interface, or the like.

In another implementation, communications interface 660 may include, for example, a transmitter that may convert baseband signals from processor 620 to radio frequency (RF) signals and/or a receiver that may convert RF signals to baseband signals. Alternatively, communications interface 660 may include a transceiver to perform functions of both a transmitter and a receiver of wireless communications (e.g., radio frequency, infrared, visual optics, etc.), wired communications (e.g., conductive wire, twisted pair cable, coaxial cable, transmission line, fiber optic cable, waveguide, etc.), or a combination of wireless and wired communications.

Communications interface 660 may connect to an antenna assembly (not shown in FIG. 6) for transmission and/or reception of the RF signals. The antenna assembly may include one or more antennas to transmit and/or receive RF signals over the air. The antenna assembly may, for example, receive RF signals from communications interface 660 and transmit the RF signals over the air, and receive RF signals over the air and provide the RF signals to communications interface 660. In one implementation, for example, communications interface 660 may communicate with network 501.

As will be described in detail below, device 600 may perform certain operations. Device 600 may perform these operations in response to processor 620 executing software instructions (e.g., computer program(s)) contained in a computer-readable medium, such as memory 630, a secondary storage device (e.g., hard disk, CD-ROM, etc.), or other forms of RAM or ROM. A computer-readable medium may be defined as a non-transitory memory device. A memory device may include space within a single physical memory device or spread across multiple physical memory devices. The software instructions may be read into memory 630 from another computer-readable medium or from another device. The software instructions contained in memory 630 may cause processor 620 to perform processes described herein. Alternatively, hardwired circuitry may be used in place of or in combination with software instructions to implement processes described herein. Thus, implementations described herein are not limited to any specific combination of hardware circuitry and software.

It will be apparent that example aspects, as described above, may be implemented in many different forms of software, firmware, and hardware in the implementations illustrated in the figures. The actual software code or specialized control hardware used to implement these aspects should not be construed as limiting. Thus, the operation and behavior of the aspects were described without reference to the specific software code—it being understood that software and control hardware could be designed to implement the aspects based on the description herein.

Even though particular combinations of features are recited in the claims and/or disclosed in the specification, these combinations are not intended to limit the disclosure of the possible implementations. In fact, many of these features may be combined in ways not specifically recited in the claims and/or disclosed in the specification. Although each dependent claim listed below may directly depend on only one other claim, the disclosure of the possible implementations includes each dependent claim in combination with every other claim in the claim set.

While various actions are described as selecting, displaying, transferring, sending, receiving, generating, notifying, and storing, it will be understood that these example actions are occurring within an electronic computing and/or electronic networking environment and may require one or more computing devices, as described in FIG. 5, to complete such actions.

No element, act, or instruction used in the present application should be construed as critical or essential unless explicitly described as such. Also, as used herein, the article “a” is intended to include one or more items and may be used interchangeably with “one or more.” Where only one item is intended, the term “one” or similar language is used. Further, the phrase “based on” is intended to mean “based, at least in part, on” unless explicitly stated otherwise.

It is to be understood that examples described herein are not limited to the specific devices, methods, conditions or parameters described and/or shown herein and that the terminology used herein is for the example only and is not intended to be limiting of the claimed invention.

In the preceding specification, various preferred embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

What is claimed is:

1. A method, comprising:
receiving, by a computing system, olfactory information; and
converting, by the computing system, the olfactory information to auditory information.

2. The method of claim 1, further comprising:
receiving, by the computing device, additional auditory information;
converting, by the computing device, the additional auditory information to other olfactory information.

3. The method of claim 1, wherein the converting the olfactory information to the auditory information includes:
analyzing the olfactory information based on a database of fragrance-sound mappings.

4. The method of claim 1, wherein the computing device is artificial intelligence (AI).
